(19) United States
(12) Patent Application Publication
(10) Pub. No.: US 2023/0069590 A1
(43) Pub. Date: Mar. 2, 2023

(54) CONCENTRATED SODIUM VALPROATE FOR RAPID DELIVERY

(71) Applicant: The Regents of the University of Michigan, Ann Arbor, MI (US)

(72) Inventors: Manjunath P. Pai, Ann Arbor, MI (US); Ben Biesterveld, Ann Arbor, MI (US); Hasan Alam, Ann Arbor, MI (US)

(21) Appl. No.: 17/792,856

(22) PCT Filed: Jan. 13, 2021
(86) PCT No.: PCT/US2021/013201

§ 371 Date: Jul. 14, 2022

Related U.S. Application Data
(60) Provisional application No. 62/961,943, filed on Jan. 16, 2020.

(51) Int. Cl. A61K 31/19; A61K 9/00; A61P 43/00

(52) U.S. Cl. CPC A61K 31/19; A61K 9/0019; A61P 43/00

(57) ABSTRACT

Provided herein are pharmaceutical compositions comprising concentrated valproic acid (e.g., >200 mg/mL) for rapid delivery of high doses (e.g., 100-200 mg/kg) of valproic acid to subjects (e.g., intraosseous, intravenous, etc.), and methods of treating emergency injuries and conditions therewith.

### CROSS-REFERENCE TO RELATED APPLICATION

The present application claims priority to U.S. Provisional Patent Application Ser. No. 62/961,943 filed Jan. 16, 2020, which is hereby incorporated by reference in its entirety.

### FIELD

Provided herein are pharmaceutical compositions comprising concentrated valproic acid for rapid delivery of high doses of valproic acid to subjects (e.g., intraosseous, intravenous, etc.), and methods of treating emergency injuries and conditions therewith.

### BACKGROUND

Injuries are the leading cause of death for young people in the United States, (Ref. 1; incorporated by reference in its entirety) and traumatic brain injury (TBI) results in 2.8 million emergency department visits per year (Ref 2; incorporated by reference in its entirety). In current military conflicts, TBI is also a significant cause of morbidity and mortality. Unfortunately, current treatments for TBI are mostly supportive in nature, without any specific therapy that can improve survival or attenuate the extent of brain damage (Ref 3; incorporated by reference in its entirety).

Valproic acid (VPA), long used as an anti-epilepsy medication, has been shown to improve survival following lethal hemorrhage, polytrauma, septic shock, ischemia-reperfusion (FR), and TBI (Refs. 4-12; incorporated by reference in its entirety). VPA is a histone deacetylase inhibitor, and the mechanism of action by which it improves survival is thought to be from its ability to cause acetylation of nuclear and cytoplasmic proteins (Refs 5, 13-14; incorporated by reference in their entireties). The current FDA approved dose is 20-60 mg/kg. The current regulatory label suggests intravenous infusion at concentrations <100 mg/mL at a minimum infusion duration of 60-minutes. Higher doses raise concerns about drug toxicity and adverse effects. Achieving higher doses requires administration of (or combination of) multiple vials of VPA (e.g., 10-20 vials). Such preparation is time consuming, particularly in the field, and may risk valuable time in a setting of clinical acuity where seconds matter. Additionally, preparation in the field as described increases risk of bacterial contamination.

### SUMMARY

Provided herein are pharmaceutical compositions comprising concentrated valproic acid (e.g., 100 mg/ml, 150 mg/ml, 200 mg/mL, 250 mg/ml, 300 mg/ml, or greater, etc.) for rapid delivery of high doses (e.g., >60 mg/kg, >100 mg/kg, etc.) of valproic acid to subjects (e.g., intraosseous, intravenous, etc.), and methods of treating emergency injuries and conditions therewith.

In some embodiments, provided herein are single-dose pharmaceutical compositions comprising 4,000 mg or greater valproic acid (e.g., 4,000 mg, 4,500 mg, 5,000, mg, 5,500 mg, 6,000, mg, 6,500 mg, 7,000, mg, 7,500 mg, 8,000, mg, 8,500 mg, 9,000, mg, 9,500 mg, 10,000, mg, 11,000 mg, 12,000, mg, 13,000 mg, 14,000, mg, 15,000 mg, or more, or ranges therebetween). In some embodiments, provided herein are single-dose pharmaceutical compositions comprising valproic acid at a concentration of greater than 100 mg/ml (e.g., 120 mg/ml, 140 mg/ml, 160 mg/ml, 180 mg/ml, 200 mg/ml, 220 mg/ml, 240 mg/ml, 260 mg/ml, 280 mg/ml, 300 mg/ml, 320 mg/ml, 340 mg/ml, 360 mg/ml, 380 mg/ml, 400 mg/ml, 420 mg/ml, 440 mg/ml, 460 mg/ml, 480 mg/ml, 500 mg/ml, or ranges therebetween). In some embodiments, a pharmaceutical composition is configured for intravenous delivery. In some embodiments, a pharmaceutical composition is configured for intraosseous delivery. In some embodiments, a pharmaceutical composition comprises a pH of 7.0-8.0 (e.g., 7.0, 7.2, 7.4, 7.6, 7.8, 8.0, or ranges therebetween). In some embodiments, a pharmaceutical composition comprises 1-20% sodium chloride (e.g., 1%. 2%, 3%, 4%, 5%, 6%, 7%, 8%, 9%, 10%, 11%. 12%, 13%, 14%, 15%, 16%, 17%, 18%, 19%, 20%, or ranges therebetween). In some embodiments, the pharmaceutical composition is stable at room temperature.

In some embodiments, provided herein are methods of treating a subject for an acute medical need comprising administering a sufficient quantity of valproic acid (VPA) formulated at a concentration of greater than 100 mg/ml (e.g., 120 mg/ml, 140 mg/ml, 160 mg/ml, 180 mg/ml, 200 mg/ml, 220 mg/ml, 240 mg/ml, 260 mg/ml, 280 mg/ml, 300 mg/ml, 320 mg/ml, 340 mg/ml, 360 mg/ml, 380 mg/ml, 400 mg/ml, 420 mg/ml, 440 mg/ml, 460 mg/ml, 480 mg/ml, 500 mg/ml, or ranges therebetween) over a time span of 180 or less minutes (e.g., <1 minute, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 20 minutes, 30 minutes, 40 minutes, 50 minutes, 60 minutes 90 minutes, 120 minutes, 150 minutes, 180 minutes, or ranges therebetween) to achieve an administered dose of at least 60 mg/kg (e.g., 60 mg/kg, 80 mg/kg, 100 mg/kg, 120 mg/kg, 140 mg/kg, 160 mg/kg, 180 mg/kg, 200 mg/kg, or more, or ranges therebetween) within the subject. In some embodiments, the acute medical need is selected from acute physical trauma, hemorrhage, traumatic brain injury, sepsis, shock, cardiac arrest, and ischemia-reperfusion injury. In some embodiments, the VPA is administered via intraosseous delivery. In some embodiments, the VPA is administered via intravenous delivery. In some embodiments, the VPA formulation is stable at room temperature. In some embodiments, the sufficient quantity of valproic acid (VPA) is contained within a single dosage unit. In some embodiments, administration is performed in the field, outside of a clinical setting. In some embodiments, administration is performed in an emergency medical setting.

### BRIEF DESCRIPTION OF THE DRAWINGS

FIG. 1: Experimental timeline. Invasive monitors were placed (cannulation), followed by femur, rectus muscle and liver injuries, then hemorrhage and traumatic brain injury (TBI). Animals were then left in shock for 1 hour, after which treatment began with normal saline resuscitation was given over 1 hour. VPA treatment was started concurrently for 2-3 hours. Autologous packed red blood cells were given 2 hours after saline resuscitation, after which animals were observed for 4 hours prior to the experiment end. PFC=prolonged field care.

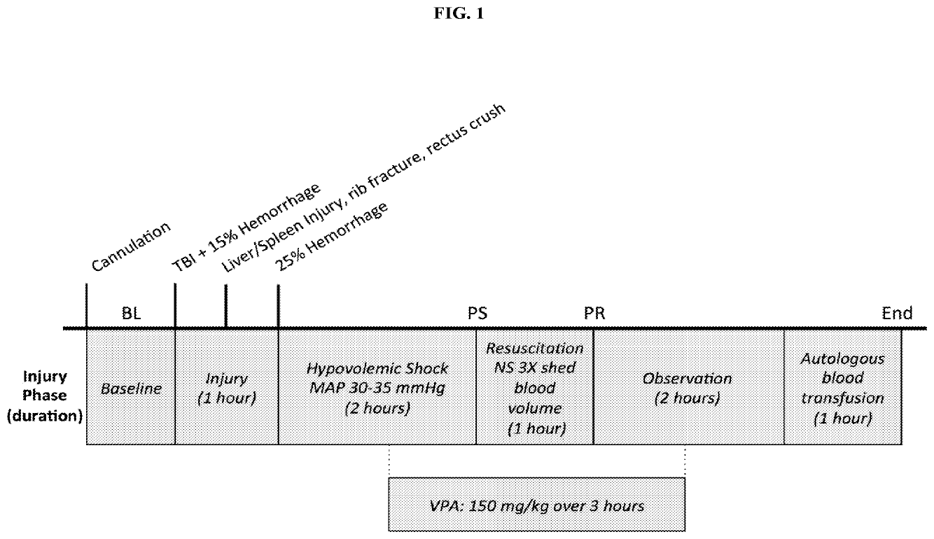

FIG. 2: Kaplan-Meier survival curves comparing survival rates. X-axis shows hours since start of VPA infusion with arrows denoting timepoints in the experiment when critical events began. * denotes prolonged survival compared to NS and VPA 100. NS=normal saline; VPA=valproic acid; pRBC=packed red blood cells.

FIG. 3: Physiologic response to injury. Intraoperative measurements of heart rate (HR) in beats per minute (BPM), mean arterial pressure (MAP) in mm Hg, cardiac output (CO) in liters per minute (L/min) and intracranial pressure (ICP) in mm Hg. X-axis shows hours since start of VPA infusion with arrows denoting timepoints in the experiment when critical events began. Error bars reflect standard deviation. ANOVA was used to compare groups at discrete timepoints. * denotes elevated MAP of VPA 150 group after treatment (p<0.05). NS=normal saline; VPA=valproic acid; pRBC=packed red blood cells.

FIG. 4: Brain lesion index. Measurement of brain lesion volume in mm³ divided by time from infliction of injury in minutes (mm³/minutes). Whisker bars reflect 5-95 percentile. ANOVA was used to compare groups. * denotes difference compared to NS group (p<0.05). Image on right shows representative 5 mm brain section and staining of lesion with 2% 2,3,5-triphenyltetrazolium chloride. NS=normal saline; VPA=valproic acid.

FIG. 5: Plasma valproic acid (VPA) concentration by treatment group. ANOVA was used to compare treatment groups. Sham animals receiving 150 mg/kg over 3 hours had higher plasma levels at all time points (p<0.05). Animals receiving 150 mg/kg over 3 h had higher plasma VPA levels at 3 and 5 hours after infusion compared to those receiving 100 mg/kg over 2 hours (p<0.05).

FIG. 6: Survival after polytrauma (left) & hemorrhage (right).

FIG. 7: Survival after sepsis.

FIG. 8: Brain lesion size after TBI (left) & Neuroseverity score (NSS) after TBI (right).

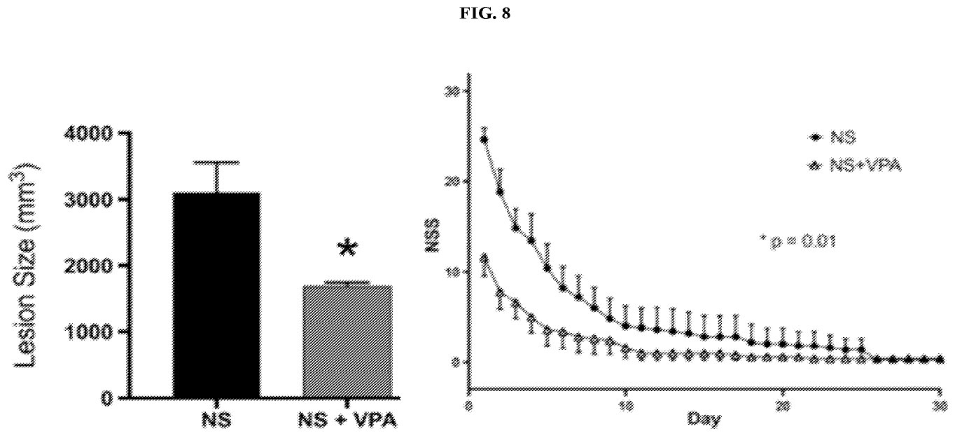

FIG. 9: Timeline. Invasive lines placed (cannulation), followed by rectus, femur and liver injuries. Animal was then flipped prone for hemorrhage and TBI. After hemorrhage, a 1-hour shock period began, followed by normal saline resuscitation. VPA infusion was started concurrent to starting NS resuscitation and lasted 3 hours. After VPA infusion, autologous packed red blood cells were given, and then the animal was monitored for an additional 4 hours prior to end of the experiment. VPA=valproic acid; PFC=prolonged field care.

FIG. 10: Serum valproic acid concentrations. Control=control group; IV=IV group; IO=IO group.

FIG. 11: Kaplan-Meier survival curves. X-axis shows hours since start of VPA infusion with important experiment timepoints denoted. Control=control group; IV=IV group; IO=IO group.

FIG. 12: Hemodynamics curves in response to injury and resuscitation. Heart rate (HR) measured as beats per minute (BPM), mean arterial pressure (MAP) measured in mmHg, cardiac output (CO) measured in liters per minute (L/min) and central venous pressure (CVP) measured in mmHg. X-axis shows hours since start of VPA infusion with important experiment timepoints denoted. Control=control group; IV=IV group; IO=IO group.

### DEFINITIONS

Although any methods and materials similar or equivalent to those described herein can be used in the practice or testing of embodiments described herein, some preferred methods, compositions, devices, and materials are described herein. However, before the present materials and methods are described, it is to be understood that this invention is not limited to the particular molecules, compositions, methodologies or protocols herein described, as these may vary in accordance with routine experimentation and optimization. It is also to be understood that the terminology used in the description is for the purpose of describing the particular versions or embodiments only, and is not intended to limit the scope of the embodiments described herein.

Unless otherwise defined, all technical and scientific terms used herein have the same meaning as commonly understood by one of ordinary skill in the art to which this invention belongs. However, in case of conflict, the present specification, including definitions, will control. Accordingly, in the context of the embodiments described herein, the following definitions apply.

As used herein and in the appended claims, the singular forms “a”, “an” and “the” include plural reference unless the context clearly dictates otherwise.

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

As used herein, the terms “treat,” “treatment,” and “treating” refer to reducing the amount or severity of a particular condition, disease state, or symptoms thereof, in a subject presently experiencing or afflicted with the condition or disease state. The terms do not necessarily indicate complete treatment (e.g., total elimination of the condition, disease, or symptoms thereof).

As used herein, the terms “prevent,” “prevention,” and preventing” refer to reducing the likelihood of a particular condition or disease state from occurring in a subject not presently experiencing or afflicted with the condition or disease state. The terms do not necessarily indicate complete or absolute prevention.

As used herein, the term “subject” broadly refers to any animal, including human and non-human animals (e.g., dogs, cats, cows, horses, sheep, poultry, fish, crustaceans, etc.). As used herein, the term “patient” typically refers to a subject that is being treated for a disease or condition.

As used herein, the term “pharmaceutical composition” refers to the combination of an active agent with a carrier, inert or active, making the composition especially suitable for diagnostic or therapeutic use in vitro, in vivo or ex vivo.

The terms “pharmaceutically acceptable” or “pharmacologically acceptable,” as used herein, refer to compositions that do not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintigrants (e.g., potato starch or sodium starch glycolate), and the like. The compositions also can include stabilizers and preservatives. For examples of carriers, stabilizers and adjuvants, see, e.g., Martin, Remington's Pharmaceutical Sciences, 15th Ed., Mack Publ. Co., Easton, Pa. (1975), incorporated herein by reference in its entirety.

As used herein, the terms “administration” and “administering” refer to the act of giving a drug, prodrug, or other agent, or therapeutic treatment to a subject or in vivo, in vitro, or ex vivo cells, tissues, and organs. Exemplary routes of administration to the human body can be through space under the arachnoid membrane of the brain or spinal cord (intrathecal), the eyes (ophthalmic), mouth (oral), skin (topical or transdermal), nose (nasal), lungs (inhalant), oral mucosa (buccal), ear, rectal, vaginal, by injection (e.g., intraosseously, intravenously, subcutaneously, intratumorally, intraperitoneally, etc.) and the like.

As used herein, the terms “co-administration” and “co-administering” refer to the administration of at least two agent(s) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s), and/or when co-administration of two or more agents results in sensitization of a subject to beneficial effects of one of the agents via co-administration of the other agent.

As used herein, the term “effective amount” refers to the amount of a composition sufficient to effect beneficial or desired results. An effective amount can be administered in one or more administrations, applications or dosages and is not intended to be limited to a particular formulation or administration route.

As used herein, the term “valproic acid” refers to a molecule with the chemical structure of:

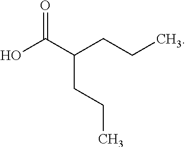

As used herein, the term “related forms,” when used in reference to valproic acid, refers to:

valproate:

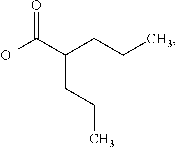

sodium valproate:

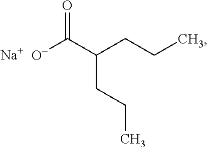

and
divalproex sodium:

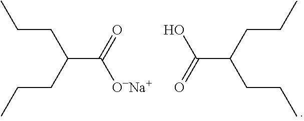

### DETAILED DESCRIPTION

Provided herein are pharmaceutical compositions comprising concentrated valproic acid (e.g., 100 mg/ml, 200 mg/mL, etc.) for rapid delivery of high doses (e.g., >60 mg/kg, >100 mg/kg, etc.) of valproic acid to subjects (e.g., intraosseous, intravenous, etc.), and methods of treating emergency injuries and conditions therewith.

In experiments conducted during development of embodiments herein escalating doses of VPA were administered in healthy volunteers, which demonstrated that doses up 140 mg/kg were well tolerated (Ref. 15; incorporated by reference in its entirety). This dose is similar to a study that showed that administration of 150 mg/kg of VPA is capable of attenuating brain lesion size and improving neurologic recovery in a swine model of non-lethal TBI and hemorrhagic shock (Refs 16-18). It has also been demonstrated that that this dose of VPA can decrease neural apoptosis, inflammation and promote neural plasticity (Ref. 19; incorporated by reference in its entirety). The dose of VPA that can improve survival in an otherwise lethal model of polytrauma, HS and TBI remains unknown in the field.

Experiments were conducted during development of embodiments herein to identify the lowest dose of VPA that improves survival in a lethal model of polytrauma, HS and TBI in swine. Experiments were conducted during development of embodiments herein to the effect of differing doses of VPA administration on survival in a clinically realistic polytrauma model in swine. It was found that VPA given in a dose of 150 mg/kg over 3 hours significantly improved survival compared to NS resuscitation, while a lower dose of 100 mg/kg over 3 hours did not. It was also found that a dose of 100 mg/kg over 2 hours improved survival when given at a faster rate of infusion; this group was chosen because it received the same dose of drug compared to the VPA 100, and it was given at a rate that matched the VPA 150 group. These results indicate that both total dose of VPA and the rate of infusion influence survival.

Experiments using extremely high doses (300-450 mg/kg) of VPA in lethal models of trauma in swine found a survival benefit. These doses were chosen based on VPA dose optimization data from a lethal model of hemorrhagic shock in rodents. However, effective doses in larger mammals are typically much lower than the small animal models (e.g. rodents). It has been demonstrated that a dose of 150 mg/kg is sufficient to cause a significant increase in histone acetylation at the end of a 3-hour VPA infusion in a swine model of combined TBI+HS.

In addition to identifying the lowest possible dose that is effective in improving survival, experiments were also conducted during development of embodiments herein to determine the effects of the rate of administration. It would be expected that rapid administration could result in hypotension in animals. In pure hemorrhage models (without TBI) with a very high early mortality, VPA was administered rapidly, and while rapid VPA administration improved survival, rapid infusion caused hypotension. In the non-lethal TBI models, to avoid hypotension which could worsen the brain injury, the drug as an infusion typically over 3 hours. In previous experiments, VPA (up to 150 mg/kg) was delivered over 1 hour without any hemodynamic adverse effects, but these subjects were healthy individuals and the results might be different in patients that are in hemorrhagic shock. Experiments conducted during development of embodiments herein demonstrate that administration of 150 mg/kg over 3 hours or 100 mg/kg over 2 hours improves survival compared to 100 mg/kg over 3 hours, without causing any adverse hemodynamic effects. Further experiments have found infusions of 150 mg/kg delivered over 1 hour also does not cause adverse hemodynamic effects.

The PK data indicates that trauma, shock and resuscitation markedly affect the plasma level of VPA. This is highlighted by the fact that the Sham VPA animals had 1.6 times higher VPA levels than the VPA 150 at the end of infusion with the same dosing regimen. The peak plasma level reported in a trial of healthy human subjects for VPA 140 mg/kg given over one hour was 1271 μg/mL. At the end of a three-hour infusion, levels in the experiments conducted herein were modest in comparison at only 291 μg/mL for Sham VPA and 175 μg/mL for VPA 150. The infusion rates were three times faster, but taken together, this data supports the need for doses of VPA that are higher than what is currently approved by the FDA for clinical use, and that these high doses are safe to administer. Differences in plasma levels of VPA were observed with the VPA 150 compared to VPA 100 over 2 h groups. However, the same differences were not observed with VPA 100. The VPA 100 group did have decreased clearance of VPA compared to other treated groups. The volume of distribution (Va.) for the VPA 150 animals was significantly higher than VPA 100, which could explain why the plasma levels were not different and help to explain why they had a marked increase in survival with more drug exiting the plasma into the tissues.

A clustering of animal deaths on the experiment timeline was around blood transfusion. Eight animals died during transfusion of pRBCs of 20 animals that survived to this point of the experiment. This is a relatively large number, and it is believed that this is related to an ischemia-reperfusion (FR) injury. VPA 150 animals were better able to tolerate this insult, which is consistent the protective effect of VPA on I/R. The sole VPA 150 animal that died, did so during the transfusion period. In this specific animal, ionized calcium dropped from 1.3 to 0.8 mmol/L abruptly during transfusion, and its cause of death could be from chelation related hypocalcemia rather than FR injury. No other animal had a similar change in calcium, so the overall clustering of animal deaths around this time point is not from transfusion related chelation and hypocalcemia.

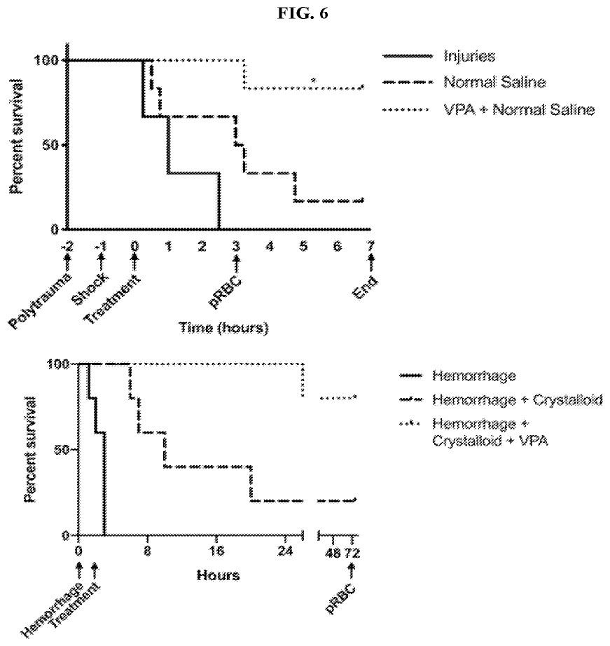

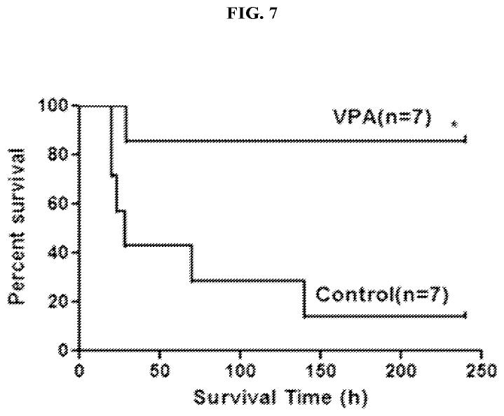

VPA is a well-known histone deacetylase (HDAC) inhibitor. A single dose of VPA administered early after injury can improve mortality in realistic large animal models of polytrauma, hemorrhage and TBI which are otherwise lethal (FIG. 6; Refs. A8-A10; incorporated by reference in their entireties). VPA also improves survival in lethal models of sepsis (FIG. 7; Refs. A11-A12; incorporated by reference in their entireties). Severe shock often leads to multisystem organ failure, and VPA attenuates this end organ injury. Specifically, a single dose of VPA attenuates kidney, heart, lung, liver and intestinal injury after hemorrhagic shock, septic shock and I-R injury (Refs, A8-A9, A13-A18; incorporated by reference in their entireties). Hemorrhagic and septic shock can also cause severe acidosis and disturbances in the coagulation cascade; VPA mitigates this acidosis, coagulopathy and platelet dysfunction (Refs. A13-A19; incorporated by reference in their entireties). A contributor to the acidosis and coagulopathy often witnessed in shock is the large volume of crystalloid resuscitation needed to achieve an acceptable blood pressure to maintain end organ perfusion; VPA has been shown to limit the crystalloid necessary to achieve and acceptable blood pressure, which contributes to the improvement in coagulopathy and acidosis (Ref. A9; incorporated by reference in its entirety). This decreased fluid resuscitation requirement also limits the resources needed to care for the patient in shock, which is of critical importance in resource scarce environments such as field care in the prehospital environment.

VPA also improves neurocognitive outcomes after TBI. In models of TBI, a single dose of VPA decreases the brain lesion size early after injury and improves brain edema (Refs. A20-A21; incorporated by reference in their entireties). VPA treatment results in improved injury scores of neuroseverity after TBI and hastens the return to normal neurologic function (Refs. A22-A23; incorporated by reference in their entireties) The beneficial effects of VPA can be seen long after injury with improved neural inflammation, apoptosis and degeneration at 30 days after injury and VPA treatment (Ref. A24; incorporated by reference in its entirety).

The mechanisms by which VPA functions are widespread. It increases the acetylation of various nuclear and cytoplasmic proteins which make the cell more able to withstand injury, shock and I/R injury. It positively affects numerous pro-survival pathways including heat shock proteins, phosphorylated-Akt, GSK-3 and β-catenin (Refs. A10, A25-A26; incorporated by reference in their entireties). Cell death and apoptosis pathways are also downregulated after VPA administration in injury (Ref. A27; incorporated by reference in its entirety).

Experiments were conducted during development of embodiments herein to demonstrate the biologic effect of IO delivered VPA. Proteomics analysis was done on selected organs thought to be critical to injury response. Significant genomic and proteomic work has been done previously examining the role of VPA in TBI (Refs. B7, B29-B32; incorporated by reference in their entireties). Many proteins differentially expressed after VPA treatment in the heart, lung and liver. A more pronounced change occurred in the lung compared to the heart and liver, with many more GO biologic processes affected.

Consistent with the TBI genomic and proteomic work, there were many GO biologic processes involved in the inflammatory response, cellular metabolism, and transcriptional & translational machinery affected, all of which indicate a cytoprotective effect. Specifically network analysis using iPathway revealed a central suite of proteins in the lung critical for protein metabolism and translation that were enriched with VPA treatment including ribosomes (RPS20, RPS28, RPL13A, RPL8, RPL21, RPLP1), translation initiation (EIF4B, EIF4E) and proteasome regulation (PSMA6, PMB3, PSMB4, PSMB8, PSMD9). The most significantly affected pathway in the lung was NIK/NF-kappaB signaling. Specific proteins in our dataset that most significantly attributed to this enriched GO term include a downregulation of programmed cell death proteins (PDCD), co-activators of NF-kB related transcription (ACTN4), phosphatases involved in cell stress response (PSMD9) and a host of proteasome subunits involved in ATP/ubiquitin-dependent peptide cleavage; taken together this indicates a general shift away from a catabolic and cell death state towards an anabolic and cell survival state. Liver and heart network analysis differed from the lung tissue, however the central suite of proteins was highly conserved between the heart and liver. This central suite in the network analysis for heart and liver included those involved in oxidative metabolism (ATP synthase subunits) and RNA transcription (LSM4, SNRPD1, SRRM2).

Experiments conducted during development of embodiments herein demonstrate that IO infusion of VPA achieves similar serum levels of VPA compared to IV infusion and this results in a similar survival benefit. The survival benefit of a single dose of VPA given early after injury has been demonstrated in a model of hemorrhage and polytrauma. Experiments herein demonstrate the noninferiority of IO VPA, particularly in emergent settings and/or with extremity injury and hypovolemia.

In some embodiments, provided herein are pharmaceutical compositions comprising valproic acid or VPA derivatives. In some embodiments, such pharmaceutical compositions are packaged and/or provided in dosages for single-dose administration. In some embodiments, each does comprises 4,000 mg or greater of valproic acid or a suitable valproic acid derivative (e.g., 4,000 mg, 4,500 mg, 5,000, mg, 5,500 mg, 6,000, mg, 6,500 mg, 7,000, mg, 7,500 mg, 8,000, mg, 8,500 mg, 9,000, mg, 9,500 mg, 10,000, mg, 11,000 mg, 12,000, mg, 13,000 mg, 14,000, mg, 15,000 mg, or more, or ranges therebetween).

In some embodiments, pharmaceutical compositions are provided as a liquid comprising VPA or a derivative thereof. In some embodiments, such a pharmaceutical composition comprises valproic acid or a VPA derivative at a concentration of greater than 100 mg/ml (e.g., 120 mg/ml, 140 mg/ml, 160 mg/ml, 180 mg/ml, 200 mg/ml, 220 mg/ml, 240 mg/ml, 260 mg/ml, 280 mg/ml, 300 mg/ml, 320 mg/ml, 340 mg/ml, 360 mg/ml, 380 mg/ml, 400 mg/ml, 420 mg/ml, 440 mg/ml, 460 mg/ml, 480 mg/ml, 500 mg/ml, or ranges therebetween). some embodiments, a pharmaceutical composition is configured for parenteral delivery. In some embodiments, a pharmaceutical composition is configured for intravenous delivery. In some embodiments, a pharmaceutical composition is configured for intraosseous delivery. In some embodiments, a pharmaceutical composition comprises a pH of 7.0-8.0 (e.g., 7.0, 7.1, 7.2, 7.3, 7.4, 7.5, 7.6, 7.7, 7.8, 7.9, 8.0, or ranges therebetween). In some embodiments, a pharmaceutical composition comprises 1-20% sodium chloride (e.g., 1%. 2%, 3%, 4%, 5%, 6%, 7%, 8%, 9%, 10%, 11%. 12%, 13%, 14%, 15%, 16%, 17%, 18%, 19%, 20%, or ranges therebetween). In some embodiments, the pharmaceutical composition is stable at room temperature. In some embodiments, the pharmaceutical composition is provided in a vial. Tube, ampule, bag, or other suitable container. In some embodiments, a single dose of a pharmaceutical composition provided herein comprises 5-100 ml of volume (e.g., 5, 10, 15, 20, 25, 30, 35, 40, 45, 50, 55, 60, 65, 70, 75, 80, 85, 90, 95, 100, or ranges therebetween).

In some embodiments, provided herein are methods of treating a subject for an acute medical need comprising administering a sufficient quantity of valproic acid or a VPA derivative formulated at a high concentration. In certain embodiment, a high concentration of VPA or a VPA derivative is greater than 100 mg/ml (e.g., 120 mg/ml, 140 mg/ml, 160 mg/ml, 180 mg/ml, 200 mg/ml, 220 mg/ml, 240 mg/ml, 260 mg/ml, 280 mg/ml, 300 mg/ml, 320 mg/ml, 340 mg/ml, 360 mg/ml, 380 mg/ml, 400 mg/ml, 420 mg/ml, 440 mg/ml, 460 mg/ml, 480 mg/ml, 500 mg/ml, or ranges therebetween). In some embodiments, a high concentration of VPA or a VPA derivative is a concentration sufficient to deliver at least 60 mg/kg (e.g., 60 mg/kg, 80 mg/kg, 100 mg/kg, 120 mg/kg, 140 mg/kg, 160 mg/kg, 180 mg/kg, 200 mg/kg, or more, or ranges therebetween) of VPA or the VPA derivative in a suitable volume (e.g., 5-100 ml (e.g., 5, 10, 15, 20, 25, 30, 35, 40, 45, 50, 55, 60, 65, 70, 75, 80, 85, 90, 95, 100, or ranges therebetween), etc.), over a suitable time span (e.g., 60 minutes or less (e.g., <1 minute, 1 minute, 2 minutes, 3 minutes, 4 minutes, 5 minutes, 6 minutes, 7 minutes, 8 minutes, 9 minutes, 10 minutes, 11 minutes, 12 minutes, 13 minutes, 14 minutes, 15 minutes, 20 minutes, 30 minutes, 40 minutes, 50 minutes, 60 minutes or ranges therebetween), etc.), and in a single dose (e.g., packaged within a single dosage).

In some embodiments, methods herein comprising delivering at least 60 mg/kg (e.g., 60 mg/kg, 80 mg/kg, 100 mg/kg, 120 mg/kg, 140 mg/kg, 160 mg/kg, 180 mg/kg, 200 mg/kg, or more, or ranges therebetween) VPA or a VPA derivative to a subject for the treatment or prevention of an acute medical need such as acute physical trauma, hemorrhage, traumatic brain injury, sepsis, shock, cardiac arrest, and ischemia-reperfusion injury. In some embodiments, VPA or a derivative thereof is administered by a parenteral route. In some embodiments, the VPA or a derivative thereof is administered via intraosseous delivery. In some embodiments, the VPA or a derivative thereof is administered via intravenous delivery. In some embodiments, the formulation is stable at room temperature. In some embodiments, the sufficient quantity of valproic acid (VPA) or VPA derivative is contained within a single dosage unit. In some embodiments, administration is performed in the field, outside of a clinical setting. In some embodiments, administration is performed in an emergency medical setting.

In some embodiments, related forms of VPA and/or VPA derivatives find use in embodiments described herein as comprising or utilizing VPA. A variety of valproic acid derivatives are well known in the field. Experiments have demonstrated that valproic acid derivatives, such as valnoctamide (VCD) and c-Butyl-propyl-acetamide (SPD), exhibit similar biological characteristics to VPA (Neuman et al. Clinical Biochemistry 46 (2013) 1532-1537; incorporated by reference in its entirety). In some embodiments herein, a valproic acid derivative is employed in place of VPA (e.g., in an embodiments herein described as utilizing VPA in a method or pharmaceutical composition). In some embodiments, provided herein are pharmaceutical compositions and methods comprising a suitable VPA derivative. In some embodiments, any embodiments described herein comprising VPA may also be employed or provided with a suitable VPA derivative. Exemplary VPA derivatives include VCD, SPD, and the VPA derivatives depicted in Table A.

TABLE A
Exemplary Valproic Acid Derivatives.

| | |
|---|---|
| 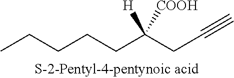 | I |
| 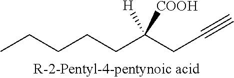 | II |
| 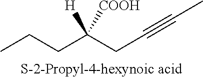 | III |
| 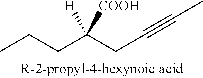 | IV |
| 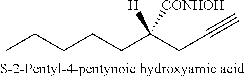 | V |
| 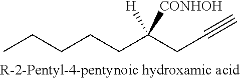 | VI |
| 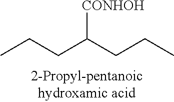 | VII |
| 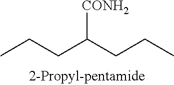 | (VPD) |
| 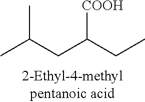 | VIII |
| 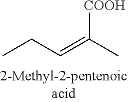 | IX |
| 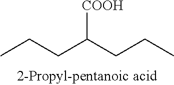 | (VPA) |

In some embodiments, a suitable VPA derivative comprises a 6-8 member alkyl chain. In some embodiments, the alkyl chain comprises one or more double or triple bonds. In some embodiments, the alkyl chain comprises (CH₂)₆, (CH₂)₇, or CH₂)₈. In some embodiments, the VPA derivative comprises a substituent at the 4 or 5 position of the alkyl chain. In some embodiments, the alkyl chain comprises a substituent at one or more additional positions.

In some embodiments, a valproic acid derivatives are branched carboxylic acids as described by Formula 1:

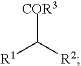

wherein R¹ and R² independently are saturated or unsaturated aliphatic C2-5, which optionally comprises one or several heteroatoms and which may be substituted, R³ is hydroxyl, halogen, alkoxy or an optionally alkylated amino group. Different R¹ and R² residues give rise to chiral compounds. The present invention encompasses the racemic mixtures of the respective compounds. The hydrocarbon chains R¹ and R² may comprise one or several heteroatoms (e.g. O, N, S) replacing carbon atoms in the hydrocarbon chain. This is due to the fact that structures very similar to that of carbon groups may be adopted by heteroatom groups when the heteroatoms have the same type of hybridization as a corresponding carbon group. R¹ and R² may be substituted. Possible substituents include hydroxyl, amino, carboxylic and alkoxy groups as well as aryl and heterocyclic groups. In some embodiments, “COR³” is a carboxylic group. In other embodiments, R³ is a halides (e.g., chloride, bromide, etc.), ester, alkoxy, etc. According to the present invention also pharmaceutically acceptable salts of a compound of formula I can be used.

Other VPA derivatives understood in the field are also within the scope herein.

The pharmaceutical formulations comprising VPA described herein can be administered to a subject by multiple administration routes, including but not limited to, oral, parenteral (e.g., intravenous, intraosseous, subcutaneous, intramuscular), intranasal, buccal, topical, rectal, or transdermal administration routes. However, parenteral route of administration, such as intravenous and intraosseous administration are particularly preferred. The pharmaceutical compositions comprising VPA described herein can be formulated into any suitable dosage form for the selected method of administration (e.g., intravenous, intraosseous, etc.).

Formulations suitable for parenteral injection may include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants. Formulations may also contain additives such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

For intravenous injections, the VPA formulations described herein may be formulated in aqueous solutions, preferably in physiologically compatible buffers such as Hank's solution, Ringer's solution, or physiological saline buffer. Penetrants appropriate to the barrier to be permeated are used in the formulation.

Parenteral injections may involve bolus injection or continuous infusion. Formulations for injection may be presented in unit dosage form, e.g., in ampoules or in multi-dose containers, with an added preservative. The pharmaceutical compositions described herein may be in a form suitable for parenteral injection as a sterile suspensions, solutions or emulsions in oily or aqueous vehicles, and may contain formulatory agents such as suspending, stabilizing and/or dispersing agents. Pharmaceutical formulations for parenteral administration include aqueous solutions of the active compounds in water-soluble form.

Rapid administration of fluids and therapeutics is essential for patients suffering from major injury, shock, bacterial sepsis, hemorrhage, trauma, anaphylaxis, etc. In practice, timely infusion of the recommended fluid volumes and therapeutic concentrations is rarely achieved due to the difficulty of obtaining stable intravenous (IV) access in the setting of critical illness or emergency situations. In some embodiments herein, particularly when IV access is difficult to obtain, a preferred technique is intraosseous (IO) administration. In some embodiments, a VPA formulation described herein is administered by an IO route. In some embodiments, a needle is drilled directly into one of the long bones the arm or leg or sternum, and fluid is administered through the bone marrow into the central circulation.

The pharmaceutical compositions comprising VPA described herein may be in unit dosage forms suitable for single administration of precise dosages. In unit dosage form, the formulation is divided into unit doses containing appropriate quantities. The unit dosage may be in the form of a package containing discrete quantities of the formulation. Non-limiting examples are packaged ampoules, vials, bottles, bags, etc. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

In some embodiments, VPA is co-administered with one or more additional therapeutics for the treatment of the conditions described herein (e.g., acute physical trauma, hemorrhage, traumatic brain injury, sepsis, shock, cardiac arrest, and ischemia-reperfusion injury).

### EXPERIMENTAL

### Example 1

### Methods

### Instrumentation and Monitoring

A 20-gauge peripheral intravenous catheter (IV) was placed in the ear for VPA administration. Bilateral 5-Fr 11-cm arterial catheters and an 8-Fr 11-cm central venous catheter (Super Sheath, Boston Scientific Corporation, Marlborough, Mass.) were placed by femoral cutdown for arterial pressure monitoring, controlled arterial hemorrhage and central venous normal saline (NS; 0.9% sodium chloride solution) administration. An open cystostomy tube was placed for urine output monitoring. The right external jugular vein was then accessed percutaneously under ultrasound guidance, and a pulmonary artery catheter was placed for monitoring of pulmonary artery pressure and cardiac output.

### Polytrauma

A 5 cm segment of the left rectus muscle was exposed and crushed using a Kelly clamp to create a soft tissue injury. Femur fracture and liver injury were performed as previously described (Ref. 20; incorporated by reference in its entirety). The mid-femur was exposed and fractured with a 22-gauge captive bolt gun (Ramset Mastershot, Glendale Heights, Ill.) fired point-blank on the femur. A reproducible grade V liver laceration was created with a liver crunch device placed 1 cm lateral to the porta and 7 cm posterior on the dome of the liver. After laceration the liver was allowed to bleed freely for 30 seconds. So as to have a reproducible injury without further uncontrolled hemorrhage, the liver was then packed with laparotomy pads (4 superior and 2 inferior to liver; 2 in each paracolic gutter), and then the abdomen was closed. Laparotomy pads were weighed at the end of the experiment to confirm a consistent liver injury (500±50 mL hemorrhage volume).

The animal was then flipped prone in preparation for TBI and additional hemorrhage. TBI was made by controlled cortical impact (Ref. 21; incorporated by reference in its entirety). A skin flap was raised on the anterior skull to expose the bregma, followed by a 21 mm burr hole on the right side next to the coronal and sagittal sutures to expose the dura. The computer controlled cortical impactor (University of Michigan Innovation Center, Ann Arbor, Mich.) was fired to produce a 10-mm depth injury. A 5-mm burr hole was made on the left for placement of the intracranial pressure (ICP) probe.

Concurrent with TBI, 40% estimated total blood volume was withdrawn over 20 minutes using a Masterflex pump (Cole-Palmer, Vernon Hills, Ill.). The initial 20% blood volume was removed over the first 7 minutes, followed by an additional 20% blood volume at a slower rate over the subsequent 13 minutes. Blood was collected into standard blood collection bags with anticoagulants (CPDA, AS-5; Terumo, Ann Arbor, Mich.) and was then centrifuged and separated into packed red blood cells (pRBCs) and plasma and stored on ice for use later in the experiment. Mean arterial pressure (MAP) was maintained between 30-35 mmHg by adjusting the inhaled isoflurane, pausing hemorrhage if MAP persisted <30 mm Hg and giving 50-100 mL fluid boluses of NS.

### Shock

After completion of hemorrhage, a one-hour shock phase began. This one hour was designed to simulate medic response time from the initial injury. During the shock phase, MAP was again maintained between 30-35 mmHg in order to standardize the effect of hypotension on the injured brain.

### Resuscitation and Treatment

After one hour of shock, animals were randomized to the following treatment groups: no resuscitation (injuries alone); NS resuscitation; or NS with VPA doses of 150 mg/kg (VPA 150) or 100 mg/kg (VPA 100) administered over 3 hours or 100 mg/kg over 2 hours (VPA 100 over 2 h). Treatment groups are outlined in Table 1. Initial VPA dosing groups were selected based upon review of existing pharmacokinetic (PK) data. A clear measure of benefit was set at a threshold survival rate of >75% in a lethal model. In order to simulate prolonged field care (PFC) by a combat medic, three times the hemorrhage volume of NS was delivered centrally through femoral vein catheter over 1 hour using the Masterflex pump. VPA was given through a peripheral IV catheter placed in the ear over 2-3 hours according to the designated treatment group. To simulate delayed evacuation to definitive care after PFC, autologous packed red blood cells (pRBCs) were transfused 3 hours after the completion of shock phase at a rate of 25 mL/hr. Animals were then monitored for an additional 4 hours to simulate time in a critical care unit, after which animals were euthanized with Euthasol (sodium pentobarbital 100 mg/kg, Virbec, Fort Worth, Tex.) if the animal survived to the end of the experiment. Laboratory parameters were monitored, and electrolyte abnormalities (e.g. hyperkalemia and hypoglycemia) were corrected as needed.

TABLE 1
Pharmacokinetic Analysis

| | | | | | Maximum | Area Under |
|---|---|---|---|---|---|---|
| | Weight | Clearance | Volume of | Half-life | Concentration | Curve |
| Animal | (kg) | (L/h) | Distribution (L) | (hrs) | (mg/L) | (h*mg/L) |
| 1 | 40 | 9.44 | 25.3 | 1.85 | 142.8 | 635.2 |
| 2 | 46 | 13.2 | 31.5 | 1.65 | 124.5 | 521.1 |
| 3 | 48 | 10.9 | 24.7 | 1.58 | 161.9 | 663.7 |
| 4 | 50 | 17.4 | 45.5 | 1.81 | 98.12 | 431.4 |
| 5 | 41 | 7.43 | 17.4 | 1.62 | 199.3 | 827.6 |
| Mean ± | 45 ± | 11.2 ± | 27.4 ± | 1.7 ± | 145 ± | 616 ± |
| SD | 3.4 | 3.2 | 8.7 | 0.12 | 38.2 | 150 |

Data are presented as group mean ± standard deviation (SD) values. Only samples from the VPA-treated animals were analyzed

A separate group of sham animals underwent cannulation and line placement without infliction of polytrauma, hemorrhage or TBI for use in subsequent pharmacokinetic (PK) analysis. These animals were given VPA 150 mg/kg over three hours (Sham VPA) and monitored for the 7 hours after the start of VPA infusion, just as other injured animals were, prior to euthanasia.

### Tissue Collection and Analysis

The brain was harvested en bloc and sliced into 5-mm coronal sections. Brain sections were then stained with 2% 2,3,5-triphenyltetrazolium chloride (Sigma Chemical, St. Louis, Mo.) to detect viable tissue. ImageJ software (National Institutes of Health, Bethesda, Md.) was then used to generate volumetric measurements of neuronal infarction to demonstrate brain lesion size. As it takes time for the brain lesion to reach its full extent, the time to death was taken into account. Brain lesion volume was evaluated as a function of time in mm³/minutes given the differences in survival times among the groups.

### Plasma VPA Levels

Blood samples were collected from the animals at 1, 3, 5 and 7 hours after the start of VPA infusion (if the animal survived to these timepoints), and at the time of death. Plasma VPA levels were determined by liquid chromatography-mass spectrometry as described previously (Ref. 18; incorporated by reference in its entirety).

### Results

### Survival Rates

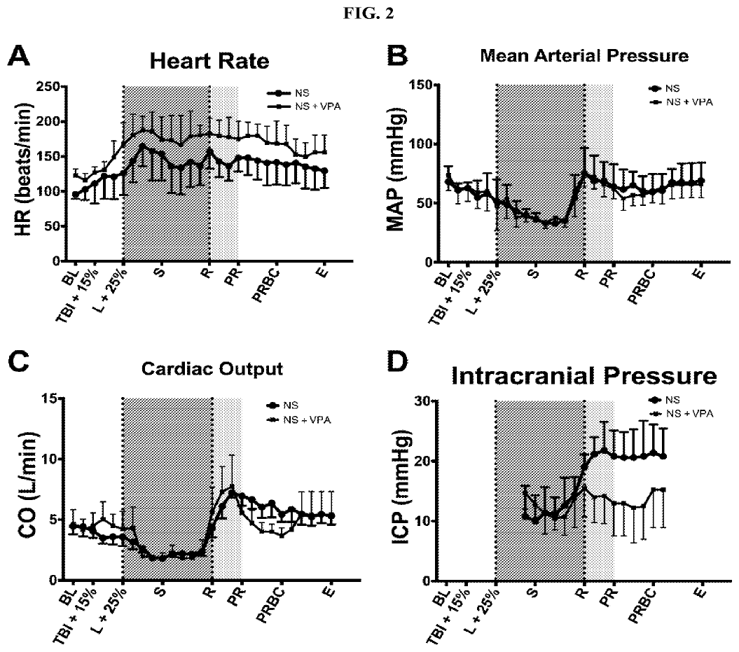

A total of 30 animals were subjected to the injuries as per protocol. Three (10%) animals died during the shock phase, prior to treatment randomization, and were not included in the final analysis. The survival curves are shown in FIG. 2. Animals that did not receive NS or VPA (injuries without treatment) died early in the experiment. NS and WA 100 group animals lived longer than those without resuscitation, and their survival curves closely mirrored each other. One NS treated animal survived to the end of the experiment, for a survival rate of 17% for the group. VPA 100 (given over 3 hours) animals had 0% survival, but VPA 100 over 2 h had 67% survival. VPA 100 over 2 h animals survived longer than VPA 100 (p<0.05), but was not statistically different than NS (p=0.07). VPA 150 had 83% survival, which was significantly different than VPA 100 and NS (p<0.05), and similar to VPA 100 over 2 h (p=0.52).

### Physiologic Parameters

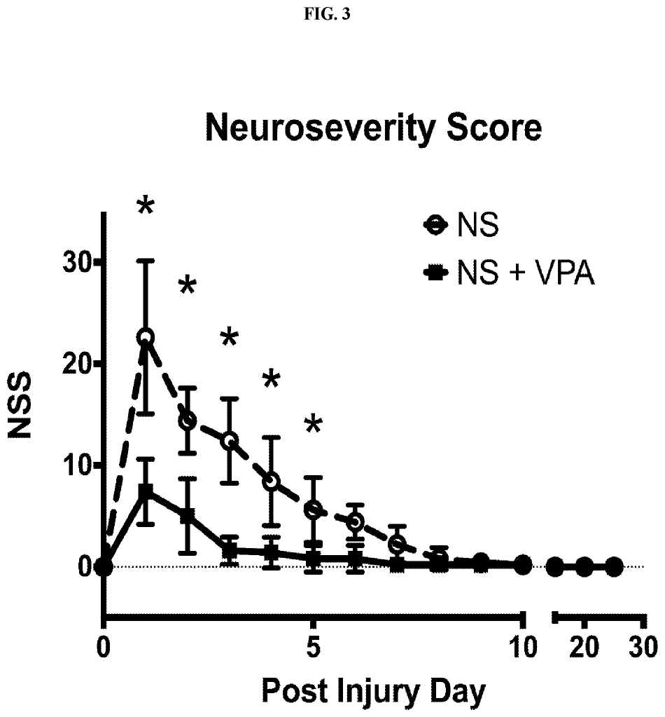

Heart rate (HR) curves did not vary between the groups. All animals became tachycardic with injuries and hemorrhage as expected. Similarly, cardiac output declined significantly during shock and increased again for all groups receiving saline resuscitation. MAP also significantly decreased with injuries and hemorrhage and improved after NS resuscitation and pRBC transfusion. Animals treated with VPA 150 tended to have a higher MAP after the treatment. Significant differences in MAP were noted compared to VPA 100 at 1 hour after the start of VPA infusion, and again at 3 hours for VPA 150 compared to all other groups (p<0.05). ICP increased throughout the experiment and did not differ between the groups (FIG. 3).

### Laboratory Values

Arterial blood gases collected serially did not demonstrate significant differences between the groups in pH, lactate, hemoglobin, sodium and potassium. Trends were noted for all groups. By the end of shock phase, lactic acidosis developed in all the groups. Hemoglobin levels declined after hemorrhage and NS resuscitation but improved following pRBC transfusion.

Serum aspartate aminotransferase (AST) levels trended to be lower in all the VPA treated groups compared to NS at 3 hours post-treatment (NS: 577±561; VPA 100: 95±29; VPA 100 over 2 h: 101±22; VPA 150: 177±95; p=0.08). No difference in the AST levels were found between any of the VPA treated groups. Group means of creatine phosphokinase (CPK) for the VPA treated animals also tended to be lower (NS 3769±3769; VPA 100: 1581±321; VPA 100 over 2 h: 927±233; VPA 150: 2428±1314). CPK level of VPA 100 over 2 h compared to NS was the only difference statistically significant (p<0.05). There were no differences between the three VPA treated groups.

### Brain Lesion

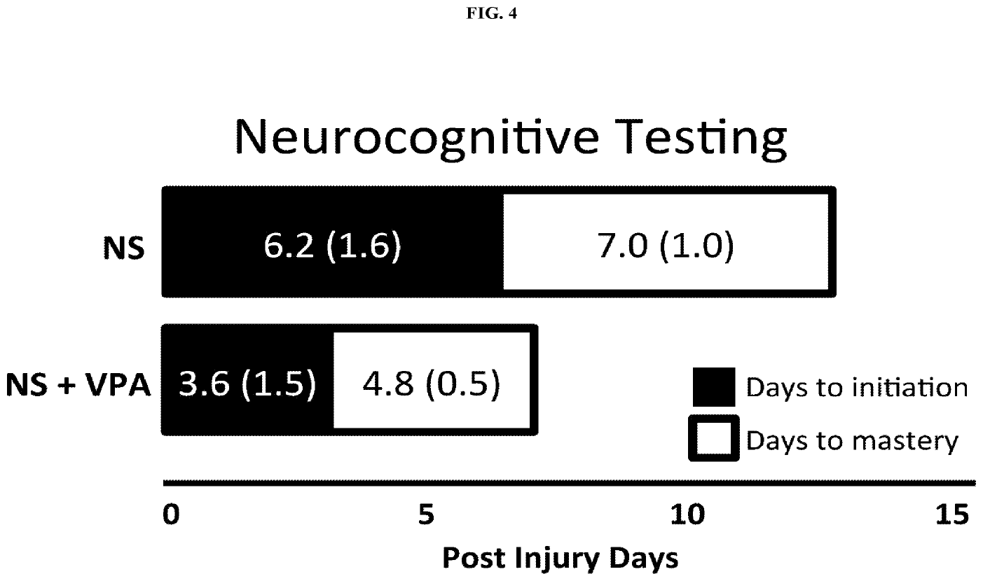

Brain lesion size at the time of necropsy positively correlated with time from the induction of TBI (p<0.05, r²=0.85). Brain lesion size not controlled for time did not differ between the groups. When controlled for time, the VPA 150 group had the smallest brain lesion at 4.1 mm³/min, which was significantly smaller than NS group that had a lesion size of 8.8 mm³/min (p<0.05) (FIG. 4). Other groups did not differ.

### Pharmacokinetic Data

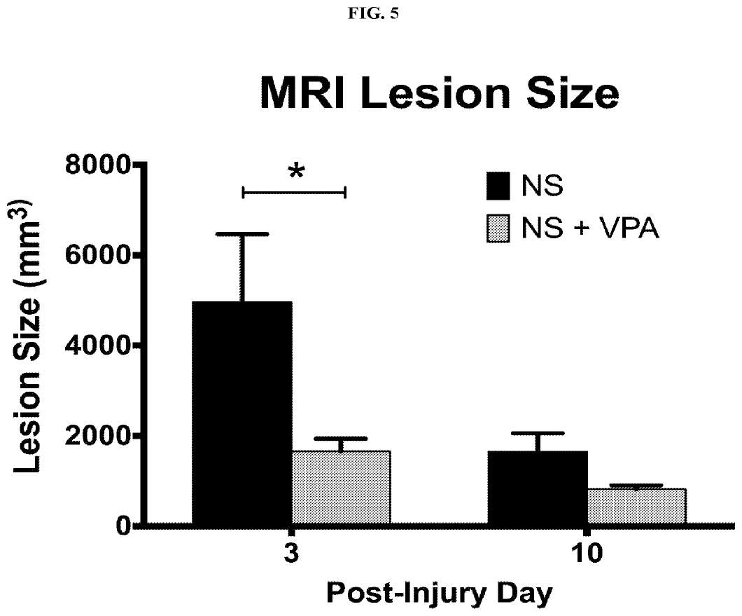

VPA levels are shown in FIG. 5. Sham VPA animals had significantly higher plasma VPA levels at 1, 3, 5 and 7 hours after start of infusion than all other groups (p<0.05). Compared to the VPA 150 group, which was given the same dose at the same infusion rate, this difference was most pronounced at the end of the infusion, three hours after the infusion had started (Sham VPA: 291 μg/mL vs. VPA 150: 175 μg/mL). For animals subjected to the trauma protocol, VPA levels were not different one hour after start of infusion. Three hours after start of VPA infusion, VPA 150 (175 μg/mL) was higher than VPA 100 over 2 h (115 μg/mL) (p<0.05). VPA 100 levels did not differ, though only 3 animals survived to this time point to be included in the data. Again, 5 hours after VPA infusion start, VPA 150 (114 μg/mL) was higher than VPA 100 over 2 h (74 μg/mL) (p<0.05). VPA 100 did not differ and had only 1 animal surviving to this timepoint to be included in the data. Levels were not different after seven hours among injured animals. Volume of distribution (Vd) was higher for VPA 150 (24.2±3.8 L) compared to VPA 100 (16.1±3.3) and Sham VPA (12.1±6.0) (p<0.05), and clearance was higher for VPA 150 (6.1±0.2) and WA 100 over 2 h (5.5±0.9) compared to VPA 100 (4.1±0.9) (p<0.05).

### Example 2

### Methods

### Injuries

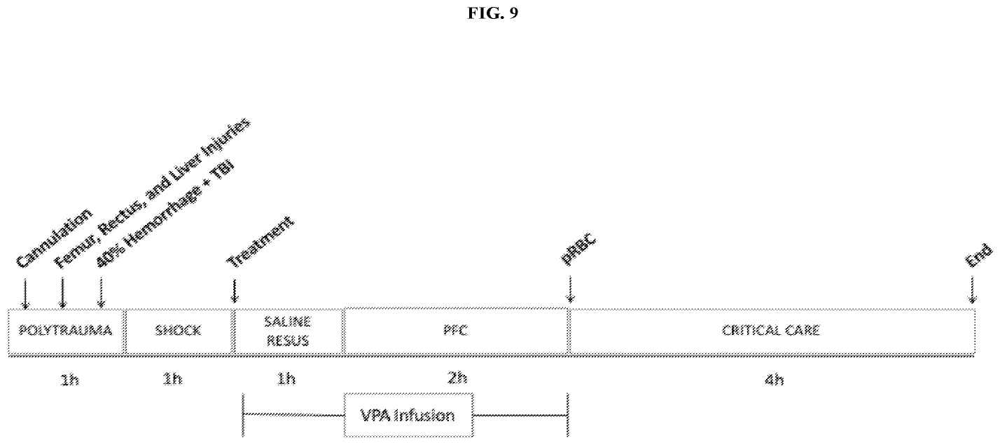

Animals underwent placement of femoral venous and arterial lines, a pulmonary artery catheter, and open cystostomy for invasive monitoring, hemorrhage and resuscitation. For the injury protocol (FIG. 9; Ref. B4; incorporated by reference in its entirety), animals had a rectus muscle crush injury on one side, femur fracture, and grade V liver laceration. The abdomen was then packed, and animal flipped prone in preparation for TBI and hemorrhage. A 12 mm depth TBI was inflicted using a controlled cortical impact device, and the animal was simultaneously hemorrhaged 40% of its estimated blood volume over 20 minutes. At the completion of hemorrhage, the animal was left unresuscitated and in shock for 1 hour to simulate medic response time.

### Resuscitation and Treatment

At the end of the shock phase, in order to simulate available resuscitative fluids available in a field care setting, all animals were resuscitated with normal saline via the femoral venous catheter with a standardized volume that was three times the hemorrhaged volume delivered over 1 hour. Animals were randomized to receive either a VPA administered IO (TO group) or administered IV (IV group) or to a control group. VPA was administered via an IO needle placed in the left humeral head; Dieckmann Intraosseous Infusion Needle, Cook Medical, Bloomington, Ind.) or via a 20-gauge peripheral IV placed in an ear vein. Placement of the needle was confirmed by aspiration of marrow and subsequent flush of 10 mL normal saline without extravasation. A dose of VPA 150 mg/kg was administered via the IO needle or IV over 3 hours. To simulate a delayed transfer to definitive care, 3 hours after the start of normal saline resuscitation, animals were transfused autologous packed red blood cells that were hemorrhaged earlier in the experiment. Animals were then monitored for another 4 hours to the predetermined end point. If they survived to the end of the experiment, animals were then euthanized with Euthasol (sodium pentobarbital 100 mg/kg, Virbec, Fort Worth, Tex.). Arterial blood gases were drawn from the femoral arterial line serially throughout the experiment.

### VPA Monitoring

Blood samples were collected at 1, 3, 5 and 7 hours after the start of VPA infusion to measure serum VPA levels. Serum VPA levels were measured using liquid chromatography-mass spectrometry (Ref. B8; incorporated by reference in its entirety).

### Proteomics

Proteomics analysis was performed on heart liver and lung samples taken at the time of necropsy. Details of proteomics sample preparation and data analysis are provided in the supplementary materials.

### Gene Ontology Enrichment

Subsequently Gene Ontology (GO) Analysis was performed using the iPathway Guide (Advaita Bioinformatics, Plymouth, Mich.) with minimum thresholds of p<0.05 and fold change of at least 1.5 (log₂ fold change 0.6). GO terms described the highest ranked biological processes in the context of terms from the Gene Ontology Consortium database (Ref. B9; incorporated by reference in its entirety). For each GO term, differentially expressed (DE) proteins from the proteomics analysis were compared to the number of DE proteins expected by chance. We set a minimum of 5 DE expressed genes for each GO term. Redundant GO terms were manually removed.

### Statistics

GraphPad Prism v8 was used to analyze data (GraphPad Software, San Diego, Calif.). A power analysis was conducted to establish the noninferiority of serum VPA concentration administered IO compared to VPA administered IV; this was the primary endpoint of the study. A noninferiority limit of 40 μg/mL was chosen based on prior dose optimization studies (Ref. B4: α=0.05, β=0.8 and standard deviation (SD)=18. This power analysis indicated n=3/group would establish noninferiority of JO compared to IV. Area under curve (AUC) was calculated for serum VPA levels, and repeated measures ANOVA was done to test differences between IV and JO at various timepoints. Kaplan-Meier curves are presented for survival data and analyzed using log-rank comparisons. Other data is presented as mean±SD, and differences were tested using a one-way analysis of variance (ANOVA) with Tukey post-hoc testing.

### Results

### Serum VPA

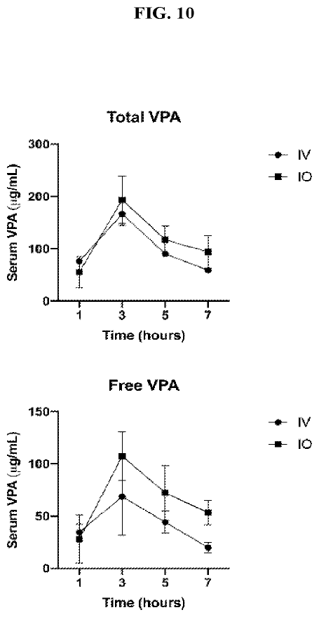

Total serum VPA AUC was similar between groups (FIG. 10). 95% confidence interval for IV was 293.5 to 355.8 h·mg/L, and for the JO group it was 301 to 469.7 h·mg/L. At the 1-, 3-, 5- and 7-hour timepoints, there was no difference between IV and IO total serum VPA (p>0.57). Free serum VPA AUC 95% confidence interval was 86.2 to 194.5 h·mg/L for the IV group and 165.3 to 275.6 h·mg/L for the JO group. At the 7-hour timepoint, the JO infusion group trended to have a higher free VPA concentration (p=0.09). At the 1-, 3- and 5-hour timepoints, free VPA was similar between both groups (p>0.59).

### Hemodynamics

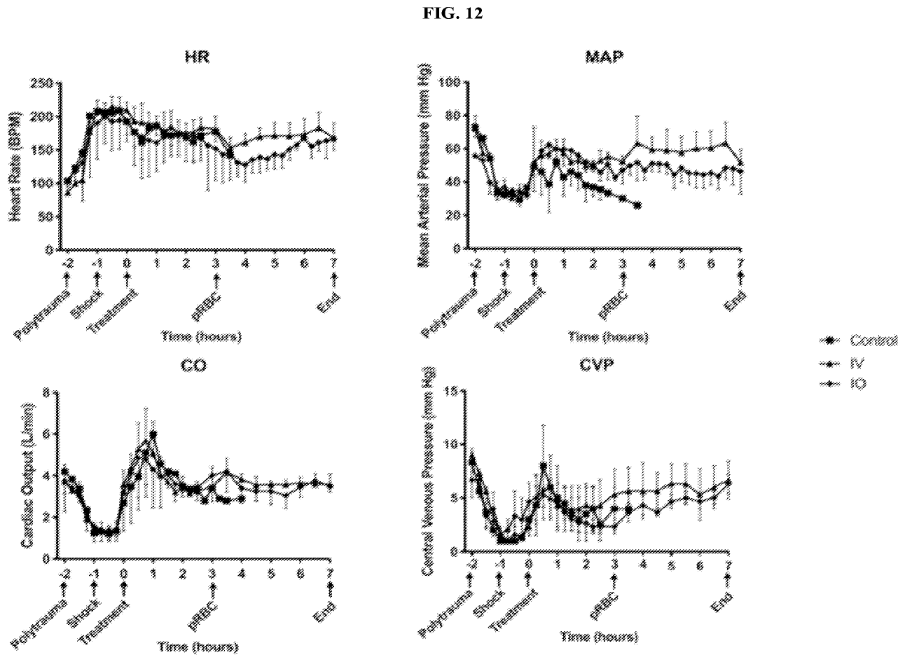

Hemodynamic curves did not vary by group and followed an expected pattern with hemorrhage and subsequent resuscitation (FIG. 12). All animals exhibited tachycardia with hemorrhage. Mean arterial pressure (MAP), cardiac output (CO) and central venous pressure (CVP) all decreased with hemorrhage and polytrauma and subsequently recovered with resuscitation.

### Arterial Blood Gas

Animals in all groups became acidotic after hemorrhage and injury as reflected by a decreasing pH, HCO₃ and increase in lactate (Table 3). There were no significant differences between groups.

TABLE 3
Arterial blood gas data. Data presented as group means with standard deviation shown
in parentheses. Hgb = hemoglobin; K = potassium; Na = sodium; HCO₃ = bicarbonate; Shock =
end of hemorrhage/start of shock; Treatment = end of shock/start of normal saline resuscitation ±
VPA treatment; pRBC = start of blood transfusion; End = end of experiment; Control = control
group; IV = VPA IV treatment group; 10 = VPA IO treatment group.

| | | Baseline | | Shock | | Treatment | | pRBC | | End | |
|---|---|---|---|---|---|---|---|---|---|---|---|
| pH | Control | 7.46 | (0.04) | 7.41 | (0.08) | 7.32 | (0.03) | 7.37 | | | |
| | IV | 7.41 | (0.06) | 7.46 | (0.02) | 7.30 | (0.18) | 7.39 | (0.05) | 7.44 | (0.07) |
| | IO | 7.46 | (0.06) | 7.48 | (0.03) | 7.37 | (0.04) | 7.39 | (0.05) | 7.41 | (0.05) |
| Hgb | Control | 9.8 | (0.9) | 10.3 | (0.8) | 10.4 | (0.8) | 6.8 | | | |
| (g/dL) | IV | 9.3 | (0.9) | 11.4 | (1.0) | 9.9 | (1.9) | 8.7 | (0.9) | 13.3 | (0.9) |
| | IO | 10.2 | (1.0) | 10.7 | (2.5) | 10.0 | (0.8) | 9.0 | (1.5) | 14.3 | (1.4) |
| K | Control | 3.7 | (0.1) | 4.7 | (1.3) | 4.6 | (1.7) | 5.2 | | | |
| (mM) | IV | 3.9 | (0.2) | 4.6 | (0.5) | 4.6 | (0.4) | 5.1 | (0.7) | 5.2 | (0.1) |
| | IO | 3.8 | (0.2) | 4.3 | (0.1) | 4.3 | (0.2) | 4.9 | (0.1) | 4.6 | (1.4) |
| Na | Control | 138 | (0.6) | 138 | (2.1) | 137 | (2.6) | 137 | | | |
| (mM) | IV | 139 | (1.0) | 135 | (3.2) | 138 | (4.6) | 140 | (1.7) | 139 | (1.0) |
| | IO | 142 | (1.2) | 139 | (0.6) | 140 | (1.7) | 143 | (1.2) | 143 | (1.2) |
| HCO₃ | Control | 26.4 | (1.5) | 18.5 | (5.9) | 13.7 | (1.8) | 15.2 | | | |
| (mM) | IV | 27.2 | (1.8) | 22.2 | (2.0) | 17.5 | (3.2) | 18.7 | (4.0) | 22.9 | (1.0) |
| | IO | 27.5 | (2.0) | 27.4 | (0.6) | 22.9 | (2.6) | 22.4 | (2.1) | 24.3 | (2.6) |
| Lactate | Control | 2.0 | (0.3) | 5.5 | (1.9) | 11.5 | (2.1) | 7.8 | | | |
| (mmol/L) | IV | 1.5 | (0.5) | 3.6 | (1.9) | 9.1 | (1.2) | 8.0 | (2.7) | 4.1 | (1.6) |
| | IO | 1.6 | (0.9) | 2.8 | (0.2) | 8.1 | (1.5) | 6.0 | (2.7) | 3.5 | (0.8) |

### Survival

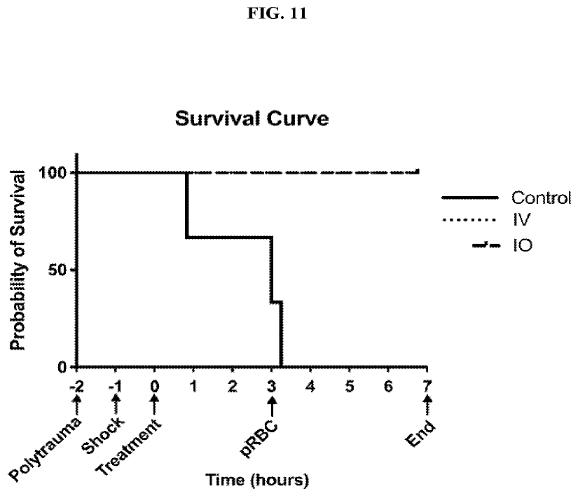

Survival is depicted in FIG. 11. Control animals had 0% survival, and both IV and IO groups had 100% survival to the end of the experiment (p<0.01).

### Proteomics

In heart tissue, there were 81 proteins DE; in lung, there were 162 proteins DE; and in liver, there were 75 proteins DE. Significantly enriched GO biologic processes are shown in Table 2. The 10 most significantly enriched GO terms for lung were selected to show for easier visualization, as 189 GO terms were significantly enriched.

TABLE 1
Gene Ontology (GO) biologic processes significantly enriched
after valproic acid treatment delivered intraosseously.
For lung, only the 10 most significantly enriched biologic
rocesses are shown, as 189 biologic processes were enriched.

| GO term | p value |
|---|---|
| Heart | |
| Positive regulation of molecular function | 0.005 |
| Regulation of catalytic activity | 0.006 |
| Inflammatory response | 0.007 |
| Regulation of hydrolase activity | 0.012 |
| Positive regulation of protein metabolism | 0.018 |
| Lung | |
| NIK/NF-kappaB signaling | 5 × 10⁻⁵ |
| Negative regulation of macromolecule | 2 × 10⁻⁴ |
| metabolic process | |
| mRNA catabolic process | 2 × 10⁻⁴ |
| Cellular macromolecule biosynthetic process | 2.8 × 10⁻⁴ |
| Negative regulation of gene expression | 4.1 × 10⁻⁴ |
| Regulation of canonical Wnt signaling pathway | 5.3 × 10⁻⁴ |
| Animal organ morphogenesis | 5.4 × 10⁻⁴ |
| SCF-dependent proteasomal ubiquitin- | 5.4 × 10⁻⁴ |
| dependent protein catabolic process | |
| Regulation of hematopoeietic stem | 6.3 × 10⁻⁴ |
| cell differentiation | |
| Anaphase promoting complex | 7.3 × 10⁻⁴ |
| Liver | 0.037 |
| Positive regulation of molecular function | 0.005 |
| Regulation of catalytic activity | 0.006 |
| Inflammatory response | 0.007 |
| Regulation of hydrolase activity | 0.024 |
| Positive regulation of protein metabolism | 0.042 |

### REFERENCES

The following references, some of which are cited above by number, are herein incorporated by reference in their entireties.
1. Rhee P, Joseph B, Pandit V, Aziz H, Vercruysse G, Kulvatunyou N, Friese R S. Increasing trauma deaths in the United States. Ann Surg. 2014; 260(1):13-21.
2. Control CfD. Traumatic Brain Injury—Related Emergency Department Visits, Hospitalizations, and Deaths—United States, 2007 and 2013. 2017. Contract No.: 9.
3. Carney N, Totten A M, O'Reilly C, Ullman J S, Hawryluk G W, Bell M J, Bratton S L, Chesnut R, Harris O A, Kissoon N, et al. Guidelines for the Management of Severe Traumatic Brain Injury, Fourth Edition. Neurosurgery. 2017; 80(1):6-15.
4. Shults C, Sailhamer E A, Li Y, Liu B, Tabbara M, Butt M U, Shuja F, Demoya M, Velmahos G, Alam H B. Surviving blood loss without fluid resuscitation. J Trauma. 2008; 64(3):629-38; discussion 38-40.
5. Gonzales E, Chen H, Munuve R, Mehrani T, Britten-Webb J, Nadel A, Alam H B, Wherry D, Burris D, Koustova E. Valproic acid prevents hemorrhage-associated lethality and affects the acetylation pattern of cardiac histones. Shock. 2006; 25(4):395-401.
6. Alam H B, Shuja F, Butt M U, Duggan M, Li Y, Zacharias N, Fukudome E Y, Liu B, Demoya M, Velmahos G C. Surviving blood loss without blood transfusion in a swine polytrauma model. Surgery. 2009; 146(2):325-33.
7. Alam H B, Hamwi K B, Duggan M, Fikry K, Lu J, Fukudome E Y, Chong W, Bramos A, Kim K, Velmahos G. Hemostatic and pharmacologic resuscitation: results of a long-term survival study in a swine polytrauma model. J Trauma. 2011; 70(3):636-45.
8. Liu Z, Li Y, Chong W, Deperalta D K, Duan X, Liu B, Halaweish I, Zhou P, Alam H B. Creating a prosurvival phenotype through a histone deacetylase inhibitor in a lethal two-hit model. Shock. 2014; 41(2):104-8.
9. Liu Z, Li Y, Liu B, Deperalta D K, Zhao T, Chong W, Duan X, Zhou P, Velmahos G C, Alam H B. Synergistic effects of hypertonic saline and valproic acid in a lethal rat two-hit model. J Trauma Acute Care Surg. 2013; 74(4):991-7; discussion 7-8.
10. Kim K, Li Y, Jin G, Chong W, Liu B, Lu J, Lee K, Demoya M, Velmahos G C, Alam H B. Effect of valproic acid on acute lung injury in a rodent model of intestinal ischemia reperfusion. Resuscitation. 2012; 83(2):243-8.
11. Causey M W, Miller S, Hoffer Z, Hempel J, Stallings J D, Jin G, Alam H, Martin M. Beneficial effects of histone deacetylase inhibition with severe hemorrhage and ischemia-reperfusion injury. J Surg Res. 2013; 184(1):533-40.
12. Jepsen C H, deMoya M A, Perner A, Sillesen M, Ostrowski S R, Alam H B, Johansson P I. Effect of valproic acid and injury on lesion size and endothelial glycocalyx shedding in a rodent model of isolated traumatic brain injury. J Trauma Acute Care Surg. 2014; 77(2):292-7.
13. Li Y, Alam H B. Modulation of acetylation: creating a pro-survival and anti-inflammatory phenotype in lethal hemorrhagic and septic shock. J Biomed Biotechnol. 2011; 2011:523481.
14. Halaweish I, Nikolian V, Georgoff P, Li Y, Alam H B. Creating a “Prosurvival Phenotype” Through Histone Deacetylase Inhibition: Past, Present, and Future. Shock. 2015; 44 Suppl 1:6-16.
15. Georgoff P E, Nikolian V C, Bonham T, Pai M P, Tafatia C, Halaweish I, To K, Watcharotone K, Parameswaran A, Luo R, et al. Safety and Tolerability of Intravenous Valproic Acid in Healthy Subjects: A Phase I Dose-Escalation Trial. Clin Pharmacokinet. 2018; 57(2):209-19.
16. Halaweish I, Bambakidis T, Chang Z, Wei H, Liu B, Li Y, Bonthrone T, Srinivasan A, Bonham T, Chtraklin K, et al. Addition of low-dose valproic acid to saline resuscitation provides neuroprotection and improves long-term outcomes in a large animal model of combined traumatic brain injury and hemorrhagic shock. J Trauma Acute Care Surg. 2015; 79(6):911-9; discussion 9.
17. Nikolian V C, Dennahy I S, Weykamp M, Williams A M, Bhatti U F, Eidy H, Ghandour M H, Chtraklin K, Li Y, Alam H B. Isoform 6-selective histone deacetylase inhibition reduces lesion size and brain swelling following traumatic brain injury and hemorrhagic shock. J Trauma Acute Care Surg. 2019; 86(2):232-9.
18. Nikolian V C, Georgoff P E, Pai M P, Dennahy I S, Chtraklin K, Eidy H, Ghandour M H, Han Y, Srinivasan A, Li Y, et al. Valproic acid decreases brain lesion size and improves neurologic recovery in swine subjected to traumatic brain injury, hemorrhagic shock, and polytrauma. J Trauma Acute Care Surg. 2017; 83(6):1066-73.
19. Chang P, Williams A M, Bhatti U F, Biesterveld B E, Liu B, Nikolian V C, Dennahy I S, Lee J, Li Y, Alam H B. Valproic Acid and Neural Apoptosis, Inflammation, and Degeneration 30 Days after Traumatic Brain Injury, Hemorrhagic Shock, and Polytrauma in a Swine Model. J Am Coll Surg. 2019; 228(3):265-75.
20. Alam H B, Bice L M, Butt M U, Cho S D, Dubick M A, Duggan M, Englehart M S, Holcomb J B, Morris M S, Prince M D, et al. Testing of blood products in a polytrauma model: results of a multi-institutional randomized preclinical trial. J Trauma. 2009; 67(4):856-64.
21. Imam A M, Jin G, Duggan M, Sillesen M, Hwabejire J O, Jepsen C H, DePeralta D, Liu B, Lu J, deMoya M A, et al. Synergistic effects of fresh frozen plasma and valproic acid treatment in a combined model of traumatic brain injury and hemorrhagic shock. Surgery. 2013; 154(2):388-96.
22. Hwabejire J O, Lu J, Liu B, Li Y, Halaweish I, Alam H B. Valproic acid for the treatment of hemorrhagic shock: a dose-optimization study. J Surg Res. 2014; 186(1):363-70.
23. Nair A B, Jacob S. A simple practice guide for dose conversion between animals and human. J Basic Clin Pharm. 2016; 7(2):27-31.
24. Georgoff P E, Halaweish I, Nikolian V C, Higgins G A, Bonham T, Tafatia C, Remmer H, Menon R, Liu B, Li Y, et al. Alterations in the human proteome following administration of valproic acid. J Trauma Acute Care Surg. 2016; 81(6):1020-7.
25. Hu S, Hou J Y, Li L, Yang M X, Sheng Z Y. [The effects of administration of valproic acid on organ function and outcome in a canine lethal hemorrhagic shock model]. Zhongguo Wei Zhong Bing Ji Jiu Yi Xue. 2011; 23(8):471-4.
A1. Centers for Disease Control and Prevention NCflPaC. Web-based Injury Statistics Query and Reporting System (WISQARS) Fatal Injury Data. 2015.
A2. Rhee P, Joseph B, Pandit V, Aziz H, Vercruysse G, Kulvatunyou N, Friese R S. Increasing trauma deaths in the United States. Ann Surg. 2014; 260(1):13-21.
A3. Davis J S, Satahoo S S, Butler F K, Dermer H, Naranjo D, Julien K, Van Haren R M, Namias N, Blackbourne L H, Schulman C I. An analysis of prehospital deaths: Who can we save? J Trauma Acute Care Surg. 2014; 77(2):213-8.
A4. Dutton R P, Stansbury L G, Leone S, Kramer E, Hess J R, Scalea T M. Trauma mortality in mature trauma systems: are we doing better? An analysis of trauma mortality patterns, 1997-2008. J Trauma. 2010; 69(3):620-6.
A5. Vincent J L, Rello J, Marshall J, Silva E, Anzueto A, Martin C D, Moreno R, Lipman J, Gomersall C, Sakr Y, et al. International study of the prevalence and outcomes of infection in intensive care units. JAMA. 2009; 302(21):2323-9.
A6. Pro C I, Yealy D M, Kellum J A, Huang D T, Barnato A E, Weissfeld L A, Pike F, Terndrup T, Wang H E, Hou P C, et al. A randomized trial of protocol-based care for early septic shock. N Engl J Med. 2014; 370(18):1683-93.
A7. Sillesen M, Bambakidis T, Dekker S E, Fabricius R, Svenningsen P, Bruhn P J, Svendsen L B, Hillingso J, Alam H B. Histone deactylase gene expression profiles are associated with outcomes in blunt trauma patients. J Trauma Acute Care Surg. 2016; 80(1):26-32; discussion −3.
A8. Biesterveld B E, Williams A M, Pai M P, Dennahy I S, Graham N J, Chtraklin K, Siddiqui A Z, O'Connell R L, Bhatti U F, Liu B, et al. Dose Optimization of Valproic Acid in a Lethal Model of Traumatic Brain Injury, Hemorrhage and Polytrauma in Swine. J Trauma Acute Care Surg. 2019.
A9. Williams A M, Bhatti U F, Biesterveld B E, Graham N J, Chtraklin K, Zhou J, Dennahy I S, Kathawate R G, Vercruysse C A, Russo R M, et al. Valproic acid improves survival and decreases resuscitation requirements in a swine model of prolonged damage control resuscitation. J Trauma Acute Care Surg. 2019; 87(2):393-401.
A10. Alam H B, Shuja F, Butt M U, Duggan M, Li Y, Zacharias N, Fukudome E Y, Liu B, Demoya M, Velmahos G C. Surviving blood loss without blood transfusion in a swine polytrauma model. Surgery. 2009; 146(2):325-33.
A11. Liu Z, Li Y, Chong W, Deperalta D K, Duan X, Liu B, Halaweish I, Zhou P, Alam H B. Creating a prosurvival phenotype through a histone deacetylase inhibitor in a lethal two-hit model. Shock. 2014; 41(2):104-8.
A12. Liu Z, Li Y, Liu B, Deperalta D K, Zhao T, Chong W, Duan X, Zhou P, Velmahos G C, Alam H B. Synergistic effects of hypertonic saline and valproic acid in a lethal rat two-hit model. J Trauma Acute Care Surg. 2013; 74(4):991-7; discussion 7-8.
A13. Causey M W, Miller S, Hoffer Z, Hempel J, Stallings J D, Jin G, Alam H, Martin M. Beneficial effects of histone deacetylase inhibition with severe hemorrhage and ischemia-reperfusion injury. J Surg Res. 2013; 184(1):533-40.
A14. Zacharias N, Sailhamer E A, Li Y, Liu B, Butt M U, Shuja F, Velmahos G C, de Moya M, Alam H B. Histone deacetylase inhibitors prevent apoptosis following lethal hemorrhagic shock in rodent kidney cells. Resuscitation. 2011; 82(1):105-9.
A15. Nikolian V C, Pan B, Mesar T, Dennahy I S, Georgoff P E, Duan X, Liu B, Wu X, Duggan M J, Alam H B, et al. Lung Protective Effects of Low-Volume Resuscitation and Pharmacologic Treatment of Swine Subjected to Polytrauma and Hemorrhagic Shock. Inflammation. 2017; 40(4): 1264-74.
A16. Zheng Q, Liu W, Liu Z, Zhao H, Han X, Zhao M. Valproic acid protects septic mice from renal injury by reducing the inflammatory response. J Surg Res. 2014; 192(1):163-9.
A17. Kim K, Li Y, Jin G, Chong W, Liu B, Lu J, Lee K, Demoya M, Velmahos G C, Alam Effect of valproic acid on acute lung injury in a rodent model of intestinal ischemia reperfusion. Resuscitation. 2012; 83(2):243-8.
A18. Williams A M, He W, Li Y, Bhatti U F, Nikolian V C, Chang P, Chang Z, Halaweish I, Liu B, Cheng X, et al. Histone Deacetylase Inhibition Attenuates Cardiomyocyte Hypoxia-Reoxygenation Injury. Curr Mol Med. 2018; 18(10):711-8.
A19. Bambakidis T, Dekker S E, Halaweish I, Liu B, Nikolian V C, Georgoff P E, Piascik P, Li Y, Sillesen M, Alam H B. Valproic acid modulates platelet and coagulation function ex vivo. Blood Coagul Fibrinolysis. 2017; 28(6):479-84.
A20. Jepsen C H, deMoya M A, Perner A, Sillesen M, Ostrowski S R, Alam H B, Johansson P I. Effect of valproic acid and injury on lesion size and endothelial glycocalyx shedding in a rodent model of isolated traumatic brain injury. J Trauma Acute Care Surg. 2014; 77(2):292-7.
A21. Jin G, Duggan M, Imam A, Demoya M A, Sillesen M, Hwabejire J, Jepsen C H, Liu B, Mejaddam A Y, Lu J, et al. Pharmacologic resuscitation for hemorrhagic shock combined with traumatic brain injury. J Trauma Acute Care Surg. 2012; 73(6):1461-70.
A22. Halaweish I, Bambakidis T, Chang Z, Wei H, Liu B, Li Y, Bonthrone T, Srinivasan A, Bonham T, Chtraklin K, et al. Addition of low-dose valproic acid to saline resuscitation provides neuroprotection and improves long-term outcomes in a large animal model of combined traumatic brain injury and hemorrhagic shock. J Trauma Acute Care Surg. 2015; 79(6):911-9; discussion 9.
A23. Nikolian V C, Georgoff P E, Pai M P, Dennahy I S, Chtraklin K, Eidy H, Ghandour M H, Han Y, Srinivasan A, Li Y, et al. Valproic acid decreases brain lesion size and improves neurologic recovery in swine subjected to traumatic brain injury, hemorrhagic shock, and polytrauma. J Trauma Acute Care Surg. 2017; 83(6):1066-73.
A24. Chang P, Williams A M, Bhatti U F, Biesterveld B E, Liu B, Nikolian V C, Dennahy I S, Lee J, Li Y, Alam H B. Valproic Acid and Neural Apoptosis, Inflammation, and Degeneration 30 Days after Traumatic Brain Injury, Hemorrhagic Shock, and Polytrauma in a Swine Model. J Am Coll Surg. 2019; 228(3):265-75.
A25. Chang P, Weykamp M, Dennahy I S, Williams A M, Bhatti U F, Liu B, Nikolian V C, Li Y, Alam H B. Histone deacetylase inhibitors: Isoform selectivity improves survival in a hemorrhagic shock model. J Trauma Acute Care Surg. 2018; 84(5):795-801.
A26. Li Y, Liu B, Sailhamer E A, Yuan Z, Shults C, Velmahos G C, deMoya M, Shuja F, Butt M U, Alam H B. Cell protective mechanism of valproic acid in lethal hemorrhagic shock. Surgery. 2008; 144(2):217-24.
A27. Butt M U, Sailhamer E A, Li Y, Liu B, Shuja F, Velmahos G C, DeMoya M, King D R, Alam H B. Pharmacologic resuscitation: cell protective mechanisms of histone deacetylase inhibition in lethal hemorrhagic shock. J Surg Res. 2009; 156(2):290-6.
A28. Weykamp M, Nikolian V C, Dennahy I S, Higgins G A, Georgoff P E, Remmer H, Ghandour M H, Alam H B. Rapid valproic acid-induced modulation of the traumatic proteome in a porcine model of traumatic brain injury and hemorrhagic shock. J Surg Res. 2018; 228:84-92.
A29. Georgoff P E, Nikolian V C, Higgins G, Chtraklin K, Eidy H, Ghandour M H, Williams A, Athey B, Alam H B. Valproic acid induces prosurvival transcriptomic changes in swine subjected to traumatic injury and hemorrhagic shock. J Trauma Acute Care Surg. 2018; 84(4):642-9.
A30. Nikolian V C, Dennahy I S, Higgins G A, Williams A M, Weykamp M, Georgoff P E, Eidy H, Ghandour M H, Chang P, Alam H B. Transcriptomic changes following valproic acid treatment promote neurogenesis and minimize secondary brain injury. J Trauma Acute Care Surg. 2018; 84(3):459-65.
A31. Georgoff P E, Nikolian V C, Bonham T, Pai M P, Tafatia C, Halaweish I, To K, Watcharotone K, Parameswaran A, Luo R, et al. Safety and Tolerability of Intravenous Valproic Acid in Healthy Subjects: A Phase I Dose-Escalation Trial. Clin Pharmacokinet. 2018; 57(2):209-19.
A32. Georgoff P E, Halaweish I, Nikolian V C, Higgins G A, Bonham T, Tafatia C, Remmer H, Menon R, Liu B, Li Y, et al. Alterations in the human proteome following administration of valproic acid. J Trauma Acute Care Surg. 2016; 81(6):1020-7.
A33. Butler F K. TCCC Updates: Two Decades of Saving Lives on the Battlefield: Tactical Combat Casualty Care Turns 20. J Spec Oper Med. 2017; 17(2):166-72.
B1. Link M S, Berkow L C, Kudenchuk P J, et al. Part 7: Adult Advanced Cardiovascular Life Support: 2015 American Heart Association Guidelines Update for Cardiopulmonary Resuscitation and Emergency Cardiovascular Care. Circulation. 2015; 132(18 Suppl 2):5444-464.
B2. Santos D, Carron P N, Yersin B, Pasquier M. EZ-IO® intraosseous device implementation in a pre-hospital emergency service: A prospective study and review of the literature. Resuscitation. 2013; 84(4):440-445.
B3. Buck M L, Wiggins B S, Sesler J M. Intraosseous drug administration in children and adults during cardiopulmonary resuscitation. Ann Pharmacother. 2007; 41(10):1679-1686.
B4. Biesterveld B E, Williams A M, Pai M P, et al. Dose optimization of valproic acid in a lethal model of traumatic brain injury, hemorrhage, and multiple trauma in swine. J Trauma Acute Care Surg. 2019; 87(5):1133-1139.
B5. Williams A M, Bhatti U F, Biesterveld B E, et al. Valproic acid improves survival and decreases resuscitation requirements in a swine model of prolonged damage control resuscitation. J Trauma Acute Care Surg. 2019; 87(2):393-401.
B6. Halaweish I, Bambakidis T, Chang Z, et al. Addition of low-dose valproic acid to saline resuscitation provides neuroprotection and improves long-term outcomes in a large animal model of combined traumatic brain injury and hemorrhagic shock. J Trauma Acute Care Surg. 2015; 79(6):911-919; discussion 919.
B7. Biesterveld B E, Pumiglia L, Iancu A, et al. Valproic Acid Treatment Rescues Injured Tissues After Traumatic Brain Injury. J Trauma Acute Care Surg. 2020.
B8. Nikolian V C, Georgoff P E, Pai M P, et al. Valproic acid decreases brain lesion size and improves neurologic recovery in swine subjected to traumatic brain injury, hemorrhagic shock, and polytrauma. J Trauma Acute Care Surg. 2017; 83(6):1066-1073.
B9. Ashburner M, Ball C A, Blake J A, et al. Gene ontology: tool for the unification of biology. The Gene Ontology Consortium. Nat Genet. 2000; 25(1):25-29.
B10. Daya M R, Leroux B G, Dorian P, et al. Survival After Intravenous Versus Intraosseous Amiodarone, Lidocaine, or Placebo in Out-of-Hospital Shock-Refractory Cardiac Arrest. Circulation. 2020; 141(3):188-198.
B11. Nolan J P, Deakin C D, Ji C, et al. Intraosseous versus intravenous administration of adrenaline in patients with out-of-hospital cardiac arrest: a secondary analysis of the PARAMEDIC2 placebo-controlled trial. Intensive Care Med. 2020.
B12. Feinstein B A, Stubbs B A, Rea T, Kudenchuk P J. Intraosseous compared to intravenous drug resuscitation in out-of-hospital cardiac arrest. Resuscitation. 2017; 117:91-96.
B13. Kawano T, Grunau B, Scheuermeyer F X, et al. Intraosseous Vascular Access Is Associated With Lower Survival and Neurologic Recovery Among Patients With Out-of-Hospital Cardiac Arrest. Ann Emerg Med. 2018; 71(5):588-596.
B14. Mody P, Brown S P, Kudenchuk P J, et al. Intraosseous versus intravenous access in patients with out-of-hospital cardiac arrest: Insights from the resuscitation outcomes consortium continuous chest compression trial. Resuscitation. 2019; 134:69-75.
B15. Nguyen L, Suarez S, Daniels J, Sanchez C, Landry K, Redfield C. Effect of Intravenous Versus Intraosseous Access in Prehospital Cardiac Arrest. Air Med J. 2019; 38(3):147-149.
B16. Granfeldt A, Avis S R, Lind P C, et al. Intravenous vs. intraosseous administration of drugs during cardiac arrest: A systematic review. Resuscitation. 2020; 149:150-157.
B17. Beaumont L D, Baragchizadeh A, Johnson C, Johnson D. Effects of tibial and humerus intraosseous administration of epinephrine in a cardiac arrest swine model. Am J Disaster Med. 2016; 11(4):243-251.
B18. Hampton K, Wang E, Argame J I, Bateman T, Craig W, Johnson D. The effects of tibial intraosseous versus intravenous amiodarone administration in a hypovolemic cardiac arrest procine model. Am J Disaster Med. 2016; 11(4):253-260.
B19. Holloway C M, Jurina C S, Orszag C J, et al. Effects of humerus intraosseous versus intravenous amiodarone administration in a hypovolemic porcine model. Am J Disaster Med. 2016; 11(4):261-269.
B20. Smith S, Borgkvist B, Kist T, Annelin J, Johnson D, Long R. The effects of sternal intraosseous and intravenous administration of amiodarone in a hypovolemic swine cardiac arrest model. Am J Disaster Med. 2016; 11(4):271-277.
B21. Wimmer M H, Heffner K, Smithers M, et al. The comparison of humeral intraosseous and intravenous administration of vasopressin on return of spontaneous circulation and pharmacokinetics in a hypovolemic cardiac arrest swine model. Am J Disaster Med. 2016; 11(4):237-242.
B22. Adams T S, Blouin D, Johnson D. Effects of tibial and humerus intraosseous and intravenous vasopressin in porcine cardiac arrest model. Am J Disaster Med. 2016; 11(3):211-218.
B23. Wong M R, Reggio M J, Morocho F R, et al. Effects of intraosseous epinephrine in a cardiac arrest swine model. J Surg Res. 2016; 201(2):327-333.
B24. Jaimovich D G, Shabino C L, Ringer T V, Peters G R. Comparison of intraosseous and intravenous routes of anticonvulsant administration in a porcine model. Ann Emerg Med. 1989; 18(8):842-846.
B25. Pollack C V, Jr., Pender E S, Woodall B N, Parks B R. Intraosseous administration of antibiotics: same-dose comparison with intravenous administration in the weanling pig. Ann Emerg Med. 1991; 20(7):772-776.
B26. Jaimovich D G, Kumar A, Francom S. Evaluation of intraosseous vs intravenous antibiotic levels in a porcine model. Am J Dis Child. 1991; 145(8):946-949.
B27. Rosetti V A, Thompson B M, Miller J, Mateer J R, Aprahamian C. Intraosseous infusion: an alternative route of pediatric intravascular access. Ann Emerg Med. 1985; 14(9):885-888.
B28. Spivey W H. Intraosseous infusions. J Pediatr. 1987; 111(5):639-643.
B29. Nikolian V C, Dennahy I S, Higgins G A, et al. Transcriptomic changes following valproic acid treatment promote neurogenesis and minimize secondary brain injury. J Trauma Acute Care Surg. 2018; 84(3):459-465.
B30. Georgoff P E, Nikolian V C, Higgins G, et al. Valproic acid induces prosurvival transcriptomic changes in swine subjected to traumatic injury and hemorrhagic shock. J Trauma Acute Care Surg. 2018; 84(4):642-649.
B31. Georgoff P E, Halaweish I, Nikolian V C, et al. Alterations in the human proteome following administration of valproic acid. J Trauma Acute Care Surg. 2016; 81(6):1020-1027.
B32. Dekker S E, Biesterveld B E, Bambakidis T, et al. Modulation of Brain Transcriptome By Combined Histone Deacetylase Inhibition and Plasma Treatment Following Traumatic Brain Injury and Hemorrhagic Shock. Shock. 2020,
B33. Granger A, Abdullah I, Huebner F, et al. Histone deacetylase inhibition reduces myocardial ischemia-reperfusion injury in mice. FASEB J. 2008; 22(10):3549-3560.
B34. Tian S, Lei I, Gao W, et al. HDAC inhibitor valproic acid protects heart function through Foxml pathway after acute myocardial infarction. EBioMedicine. 2019; 39:83-94.
B35. Nagata S, Marunouchi T, Tanonaka K. Histone Deacetylase Inhibitor SAHA Treatment Prevents the Development of Heart Failure after Myocardial Infarction via an Induction of Heat-Shock Proteins in Rats. Biol Pharm Bull. 2019; 42(3):453-461.
B36. Wu S Y, Tang S E, Ko F C, Wu G C, Huang K L, Chu S J. Valproic acid attenuates acute lung injury induced by ischemia-reperfusion in rats. Anesthesiology. 2015; 122(6):1327-1337.
B37. Liu B, Qian J, Wang Q, Wang F, Ma Z, Qiao Y. Butyrate protects rat liver against total hepatic ischemia reperfusion injury with bowel congestion. PLoS One. 2014; 9(8):e106184.
B38. Qiao Y L, Qian J M, Wang F R, Ma Z Y, Wang Q W. Butyrate protects liver against ischemia reperfusion injury by inhibiting nuclear factor kappa B activation in Kupffer cells. J Surg Res. 2014; 187(2):653-659.
B39. Dokmanovic M, Clarke C, Marks P A. Histone deacetylase inhibitors: overview and perspectives. Mol Cancer Res. 2007; 5(10):981-989.
B40. Marks P A, Miller T, Richon V M. Histone deacetylases. Cuff Opin Pharmacol. 2003; 3(4):344-351.

1. A method of treating a subject for an acute medical need comprising administering a sufficient quantity of valproic acid (VPA) formulated at a concentration greater than 100 mg/ml over a time span of 180 minutes or less to achieve an administered dose of at least 100 mg/kg within the subject.

2. The method of claim 1, wherein the acute medical need is selected from acute physical trauma, hemorrhage, traumatic brain injury, sepsis, shock, cardiac arrest, and ischemia-reperfusion injury.

3. The method of claim 1, wherein the VPA is formulated at a concentration of at least 200 mg/ml.

4. The method of claim 3, wherein the VPA is formulated at a concentration of at least 300 mg/ml.

5. The method of claim 1, wherein the VPA is administered over a time span of 60 minutes or less.

6. The method of claim 5, wherein the VPA is administered over a time span of 20 minutes or less.

7. The method of claim 1, wherein the VPA is administered via intraosseous delivery.

8. The method of claim 1, wherein the VPA is administered via intravenous delivery.

9. The method of claim 1, wherein the VPA formulation is administered in sufficient quantity and concentration to achieve an administered dose of at least 100 mg/kg within the subject.

10. The method of claim 1, wherein the VPA formulation is administered in sufficient quantity and concentration to achieve an administered dose of at least 150 mg/kg within the subject.

11. The method of claim 1, wherein the VPA formulation is stable at room temperature.

12. The method of claim 1, wherein the sufficient quantity of valproic acid (VPA) is contained within a single dosage unit.

13. The method of claim 1, wherein administration is performed in the field, outside of a clinical setting.

14. The method of claim 1, wherein administration is performed in an emergency medical setting.
